(19) United States
(12) Patent Application Publication
(10) Pub. No.: US 2024/0180662 A1
(43) Pub. Date: Jun. 6, 2024

(54) DEPOSITION DEVICE FOR MEDICAL GUIDE WIRES OR CATHETERS

(71) Applicant: Loumedic GmbH, Recherswil (CH)

(72) Inventors: Levent Kara, Zürich (CH); Mario Della Morte, Schnaus (CH)

(21) Appl. No.: 18/438,837

(22) Filed: Feb. 12, 2024

Related U.S. Application Data
(63) Continuation of application No. 17/616,235, filed on Dec. 3, 2021.

(30) Foreign Application Priority Data
Jun. 7, 2019 (CH) 00779/19

(51) Int. Cl. A61B 50/33

(52) U.S. Cl. CPC A61B 50/33

(57) ABSTRACT

A deposition device for at least one wound medical guide wire or catheter. A base plate at a plate upper side defines a deposition plane. A plurality of guide elements is present projecting from the base plate to the plate upper side, the guide elements forming a first, second and third contact surface for the guide wire or catheter. The first, second and third contact surface are protruding in a section perpendicular to the deposition plane. They are arranged and orientated such that a curved path for the wound guide wire or catheter results, wherein the wound guide wire or catheter, when it is led along the path, abuts on the first, second and third contact surface and the first and third contact surface are arranged at the outside and the second contact surface at the inside of the path.

### CROSS-REFERENCE TO RELATED APPLICATION

This application is a continuation of U.S. Ser. No. 17/616,235 filed Dec. 3, 2021, which is a National Stage Application of PCT/EP2020/065608 filed Jun. 5, 2020, which claims priority to CH 00779/19 filed Jun. 7, 2019, the disclosures of which are expressly incorporated herein in their entireties.

### BACKGROUND OF THE INVENTION

### Field of the Invention

The invention relates to a deposition device for medical guide wires or catheters.

### Description of Related Art

Medical guide wires are used in urology, interventional radiology, cardiology, heart surgery, vessel surgery and gastroenterology and in further medical fields. In particular, they serve for the guiding and stabilising of catheters given their introduction. Guide wires have different stiffnesses and can furthermore be quite long, for example up to 3 m. Their deposition and the reliable storage before and during the respective medical operation is a challenge due to the demanded flexibility. The same applies to the deposition and the keeping of catheters which are likewise provided in different lengths and with different stiffnesses.

Traditionally, guide wires are deposited into a container which is filled with some saline solution. This is not entirely unproblematic in view of the intrinsic stiffness of the guide wires and in particular is also unsatisfactory if more than one guide wire is necessary and lies in the container.

For example, containers which are provided with holding elements, by way of which guide wires that are deposited in the containers are held in a defined manner at locations provided for this are known from US 2021/0312703, U.S. Pat. No. 5,769,222 and WO 2008/139852. The containers are designed for containing a liquid. The guide wires are deposited on the base of the containers in a wound manner. Above the guide wires, the holding elements form a flange-like bead, whose lower side runs parallel to the base and prevents the guide wires from being able to inadvertently jump out of the holder.

The deposition as well as removal of the wound guide wires from such containers can only be effected by way of a two-handed intervention and only with a certain effort. On depositing, the size of the windings is set in a quite restricted manner due to the design of the containers, which indeed with a repeated use of a guide wire is not unproblematic in the course of an operation which takes place under time pressure.

U.S. Pat. No. 4,332,322 shows a folding container for sterile flexible tubes. The folding container includes structures that can be folded out and that form a receiver for the flexible tubes and can thus accordingly fix these.

US 2017/0333668 shows a catheter package with a straightening device, through which the catheter is pulled on removal.

JP 2012-70905 shows deposition devices for surgical wires, amongst other things with eight paths, which are 8-shaped.

### SUMMARY OF THE INVENTION

It is the object of the invention to provide a deposition device for medical guide wires or catheters that overcomes the disadvantages of the state of the art and that, in particular, provides a simple handling and is very well suited for a repeated depositing and removal again of one or more of the guide wires or catheters.

According to an aspect of the invention, a deposition device for medical guide wires or catheters is provided includes:
a base plate which defines a deposition plane;
a plurality of guide elements projecting from the base plate;
wherein the guide elements form a first, second and third contact surface which, in a section perpendicular to the deposition plane, form a protrusion (overhang; protuberance),
and wherein the first, second and third contact surface are arranged and orientated such that a curved path (approximately parallel to the contact surface) for a guide wire or catheter that abuts on the first, second and third contact surface results, and wherein this curved path is designed such that the first and third contact surface are arranged at the outside and the second contact surface at the inside of the path.

Generally, a path in the context of the present text is a path that is assumed by a wire winding which, on account of its intrinsic stiffness, strives for a minimal curvature, i.e., a wire winding of a wire that is stiff per se and is straight in the relaxed state.

The design with the three mentioned contact surfaces has a series of advantages. Medical guide wires and catheters on account of their intrinsic stiffness have a tendency to counteract a bending by way of an elastic counter force. For this reason, it is known per se to deposit guide wires in windings in a manner limited by way of guide means, which are at the outer side, the windings on account of the outer-side limitation assuming a roughly elliptical shape. In accordance with the design, corresponding deposition devices according to the state of the art set the necessary size of the winding in a relatively precise manner. This can be disadvantageous, in particular for the re-deposition after use for the first time, given the often time-critical conditions during an operation. Furthermore, deposition devices from the state of the art require relatively much space. Finally, the removal of the deposited guide wire or catheter is also somewhat difficult, since one must take care that the guide wire or catheter does not tangle somewhere or even fall to the ground and can therefore no longer be used. In contrast to this, the design according to the invention is very simple and flexible. The elasticity of the wound guide wire causes this to seek a circular as possible configuration, which is why from a certain winding minimal length on account of its elasticity it is pressed outwards against the first and third contact surface and simultaneously inwards against the first contact surface, which is why it is held in a stable manner. Inasmuch as the winding minimal length is reached, this is the case independently of the size of the winding, thus functions for completely different winding lengths, wherein the wound guide wire or catheter follows a roughly elliptical or also pear-shaped path depending on the length of the winding and the intrinsic stiffness.

The removal is also particularly simple. A pressing-together of the guide wire winding (or catheter winding) away from the first and the third contact surface also straight way releases the guide wire or catheter from the second contact surface (the winding becomes narrower and by way of this automatically longer), so that the guide wire or catheter can be removed with a single hand intervention, under certain circumstances even with one hand.

The curvature of the path can be roughly monotonous. In particular, the path can be roughly elliptical. A roughly elliptical path automatically results depending on the length of the winding due to the intrinsic stiffness of the guide wire or catheter, when the guide wirc winding (or catheter winding) is pressed together laterally at two points (corresponding to the first and third contact surface if these are roughly point-like); the inner-lying second contact surface can effect a certain deviation from the elliptical course. The monotonous curvature of the path does not rule out the wound guide wire or catheter being deflected slightly inwards locally at the location of the abutting on the respective outside contact surface and thus being curved in a somewhat flattened manner or even locally counter to the path curvature. With the longer windings, the path can also assume a roughly pear-shaped course; the precisely resulting shape is that at which the potential energy, which is to be brought into the guide wire or catheter by way of its bending onto the path, is minimised.

Given the deposition of the wound guide wire or catheter along a curved path, this intrinsic stiffness of the guide wire or of the catheter is utilised by way of the wind guide wire or catheter tending to assume a configuration with a low as possible curvature, i.e., given a fixed winding length tending to assume a circular configuration. In the deposition device according to the invention, this is utilised by way of an intrinsically stable configuration resulting, as explained above, in which configuration the guide wire or catheter is pressed outwards against the first and third contact surface and inwards against the second contact surface, independently of the precise length of the guide wire winding or catheter winding.

At the location of the contact surfaces and in general, the guide wire or catheter is guided at only one side by the deposition device, in contrast to a guiding in the guide channel. This means that no element that inhibits the movement of the guide wire or catheter along the deposition plane is present lying opposite the respective contact surface.

The first, second and third contact surface in particular can be formed each by a first, second and third guide element, which projects from the base plate. Its arrangement is then such that the path leads past the first guide element at the inside, past the second guide element at the outside and past the third guide element at the inside.

The arrangement of the guide elements defines a front side and a rear side of the deposition device: the first and the third guide element and the contact surfaces formed by them are arranged at the front side, the second guide element accordingly at the rear side.

In order for the advantages of the invention to come to the forefront, in particular the following optional features, which can be combined with one another, can be advantageous:
At least the second guide element (and possibly also a further, for example fourth guide element, see below), past which the path is led at the outside is arranged at a distance to the edge of the base plate, i.e., the base plate extends in all directions from this guide element and, for example, also from the other guide elements.
The base plate is free of a wall at least at the rear side and/or front side, i.e., it runs out at the edge. Due to the absence of a front-side limitation, the flexibility of the device with respect to the length of the winding is completely assisted. The absence of a rear-side limitation simplifies the placing-in and removal of the wire or catheter by way of the loop, which is formed by it only having to be pressed together against its intrinsic stiffness along its smaller axis, which slightly increases the extension along its larger axis.
The distance of the guide elements is sufficiently large in order to permit a deposition without too large deformations. The minimum distance of adjacent guide elements is, for example, at least 1.5 cm or at least 2 cm or at least 3 cm or at least 4 cm. In addition or as an alternative, the distance can be larger than an average horizontal diameter of the respective guide elements, for example by at least a factor of 1.5 or at least a factor of 2.
The guide elements are designed as elements that project from the base plate in a mushroom-like manner and are fixedly connected to this. Mushroom-like means that they get thicker upwards from a thinner trunk, i.e., in the direction away from the base plate.
The contact surfaces for the guide wire or catheter form a single-sided contact—i.e., the guide elements are not designed for a clamping or the like. In contrast, the device is designed such that nothing stands in the way of a moving of the guide wire or catheter in the horizontal direction away from the contact surface. In the environment of each contact surface (perpendicularly away from the contact surface in the horizontal direction away), the base plate is free of upwardly projecting elements, so that the laying-on as well as designated removal of the winding is not inhibited in the manner which is hereinafter to be dealt with in more detail.

As a whole, the deposition device in particular is designed as an object that is intrinsically rigid and that forms the base pate and the plurality (for example, at least three) of guide elements that project perpendicular upwards therefrom or are designed in a mushroom-like manner, wherein the base plate is free of an enclosure. The fact that the deposition device is an object that is intrinsically rigid also means that it is free of moving elements, such as rollers or the like.

In particular, the deposition device can consist of a base plate and the guide elements as well as a fastening device (adhesive or the like), which is possibly present on the base plate on the lower side, i.e., can be free of further elements. The deposition device can be designed as one piece, for example as a plastic part, for example as an injection moulded part.

The number of the guide elements, for example, is at least three or at least five, for example precisely five. It would be possible per se for the first and third contact surface to be formed by a common outer guide element, which runs regionally around the path. The design with dedicated outer first and third guide element, which are separate from one another, is, however, particularly advantageous. Firstly, it is particularly material-saving and takes into account the fact that per contact surface an essentially point-contact-like bearing of the guide wire winding or catheter winding is sufficient, i.e., one needs no extended contact surface but the wound guide wire or catheter contacts the contact surface in each case only at one point. Secondly, the depositing and removal of the medical guide wire or catheter is particularly simple if movements of the guide wire or catheter along the deposition plane are possible in an uninhibited as possible manner with the exception of the contact surfaces.

For a similar reason, it can be advantageous if the contact surfaces in a section parallel to the deposition plane are convexly curved, which concerning the first and the second contact surface means that they are curved counter to the curvature of the path.

Particularly advantageously, the guide elements are designed as elements that project from the base plate in a mushroom-like manner.

On account of the protrusion (i.e., undercut with respect to directions perpendicular to the deposition plane) a for example fillet-like deepening is formed at each contact surface, in which deepening the wound guide wire or catheter is pressed on account of its intrinsic stiffness. In embodiments, at least with the front-side contact surfaces (the first and the third contact surface), this deepening is arranged at a vertical distance to the plate upper side, i.e., the guide wire or catheter is led above the plate plane and can be simply gripped from below; the distance of the deepening to the plate upper side, for example, with the front side guide element can be at least approx. 0.6 cm or at least approx. 1 cm. For this purpose, the respective guide element can include a portion that runs out to the bottom.

In the section perpendicular to the deposition plane, the guide element in particular can be concavely curved, so that the mentioned fillet forms and the guide wire or catheter obtains a defined vertical position.

The deposition device in particular can also be designed for the locally separate deposition of more than one guide wire or catheter (this includes the possibility of depositing a guide wire and catheter in each case). For this purpose, the device can form a fourth, fifth and sixth contact surface.

Herein, the third and fourth contact surface can be formed by a common guide element. Such a common guide element perpendicularly to the path direction can have a greater extension than guide elements with only one contact surface. At all events, the distance of the contact surfaces can be selected such that the paths of adjacently deposited wires or catheters do not overlap. In this context, it can also be advantageous for the main axes of the adjacent paths to form an angle to one another, so that they run away from one another to the rear, thus away from the contact surfaces.

The defined position of the paths of adjacently deposited guide wires or catheters, possibly also in the vertical direction, on removal of one of the guide wires or catheters helps to prevent the adjacently deposited guide wire or catheter from being inadvertently lifted out of the fastening on removal of one of the guide wires or catheters.

In embodiments, the deposition device includes an adhesive on the plate lower side. By way of this, it can be fastened to a surface (working surface) of a working table. This means, which retrospectively seems very simple, entails a further design advantage. It permits the possibility of not having to deposit the complete winding of the guide wire or catheter on the base plate. In contrast the guide wire or catheter can extend over the working surface of the working table. For this reason, the deposition device as whole can be designed in a very small and compact manner without compromising its functionality.

The deposition device, as already indicated further above, in particular in contrast to devices of the state of the art can be designed such that it is not container-like with peripheral lateral walls. In contrast, the deposition device in embodiments in particular can be open to the front, i.e., to the side away from the guide element that forms the second contact surface, so that the length of the winding is not limited.

In embodiments, the base plate as a whole forms an arc, i.e., it is curved in the deposition plane and specifically such that as mentioned above, the axes of the paths form an angle to one another and run away from one another to the rear, this away from the contact surfaces.

The deposition device can be transparent at least in the region of the base plate and for example as a whole.

Apart from the deposition device, which is described here in the mentioned design possibilities, the invention also relates to its use for the deposition of a medical guide wire or catheter. This is particularly authorised for medical operations and is manufactured of material that is authorised for medical operations, for example of stainless steel (bare or coated, for example with PTFE) or of nitinol (bare or coated, for example with PTFE). The deposition is effected as designated, by way of winding on the guide wire or catheter, slight lateral pressing together of the winding until the guide wire or the catheter can be inserted between the first and the third contact surface, with the guide element that forms the second contact surface on the inner side of the winding, and letting go into the intrinsically stable configuration, in which the winding on account of its intrinsic stiffness is pressed outwards onto the first and third contact surface and inwards onto the second contact surface. In embodiments with a fourth, fifth and sixth contact surface, the same accordingly also applies to a second winding which, by way of a slight lateral pressing of the winding, the catheter can be introduced between the fourth and the sixth contact surface, with the guide element which forms the fifth contact surface on the inner side of the winding and letting go into an intrinsically stable configuration, in which the winding on account of its intrinsic stiffness is pressed outwards onto the fourth and sixth contact surface and inwards onto the fifth contact surface.

In order to ensure designated use, the deposition device can be provided in a set, which apart from the deposition device also includes information—for example in paper form or electronically—in which the mentioned designated deposition is taught and in particular the arrangement of the winding with respect to the first, second and third contact surface (and possibly fourth fifth and sixth contact surface).

A designated use also includes the possibility of adding a second or, for example, also a third deposition device and thus the use of several deposition devices on one and the same working surface (working table or the like), for example next to one another.

### BRIEF DESCRIPTION OF THE DRAWINGS

Embodiments of the invention are described in more detail by way of drawings. In the figures, the same reference numerals designate the same or analogous elements. There are shown in:

FIG. 1 a view of a deposition device according to the invention;

FIG. 2 a view of the deposition device of FIG. 1 with drawn possible first and second paths:

FIG. 3 a lateral view of the deposition device according to claim 1; and

FIG. 4 a further view.

### DETAILED DESCRIPTION OF THE INVENTION

In the subsequent description of one of embodiment examples, the use for the deposition of medical guide wires is illustrated. The drawn and described deposition device however can also be used for the deposition of a catheter (possibly in combination with the deposition of a guide wire) or of several catheters.

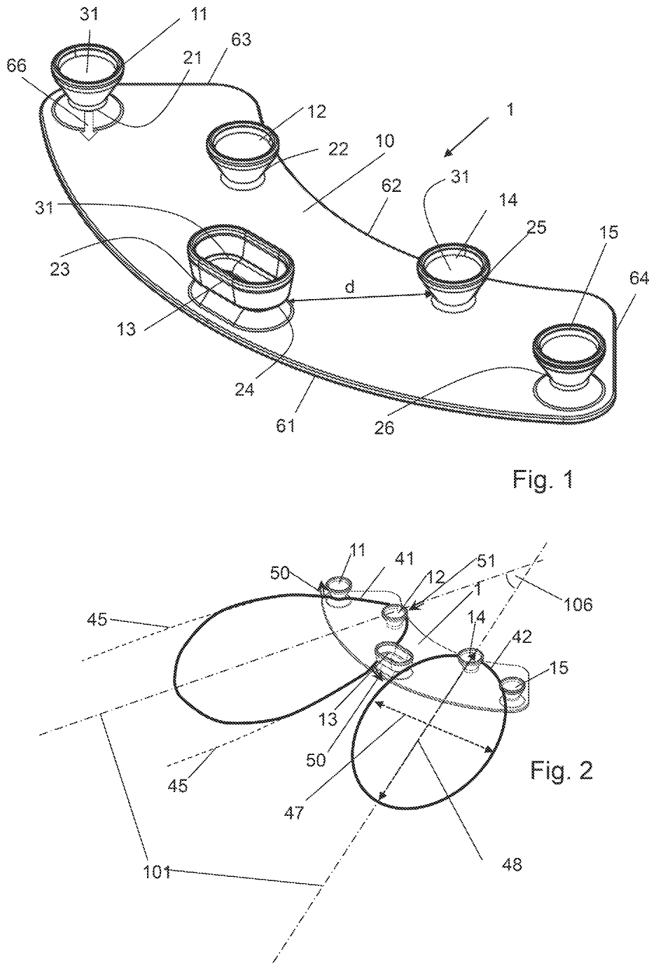

FIG. 1 shows a view of a deposition device 1 according to the invention. This includes a base plate 10 which, at the upper side, defines a deposition plane for one or more guide wires. In total five projections are formed projecting from the base plate, the projections forming first, second, third, fourth and fifth guide elements 11, 12, 13, 14, and 15. The guide elements 11-15 are each designed such that they form a protrusion at least in the region of a contact surface and thus prevent the sliding-away to the top of a wire, which abuts upon the contact surface.

As a whole, the deposition device is manufactured as a plate with a discrete number of guide elements, which project from the plate in a mushroom-like manner. It is free of a peripheral wall, i.e., is free of an enclosure.

The arrangement of the guide elements defines a front side and a rear side, wherein the first, third and fifth guide element 11, 13, 15 are arranged at the front side and the second and fourth guide element at the rear side with respect to this. The guide elements are arranged at a distance to the edges 61, 62, 63, 64, wherein, in particular, a distance of the second and fourth guide element 12, 14 to the rear edge 62 is significant. Likewise, significant is the fact that at least at the front or rear no walls are present, so that the windings can be easily deposited and removed again.

The guide elements are fixedly and rigidly connected to the base plate, so that the deposition device is always stable and cannot displace, even when working under time pressure and possibly cumbersome movements. As a whole, the deposition device in particular is manufactured as a monolithic object, for example of plastic, for example as an injection moulded part.

The distances d of adjacent guide elements—in FIG. 1 by way of example the distance between the third and the fourth guide element is drawn—are selected such that the deposition, which is described in more detail hereinafter, is possible. It is typically a few centimetres, in particular at least 2 cm.

FIG. 2 illustrates a first path 41 and a second path 42, which is defined by the deposition device and along which a wound guide wire can be deposited. The windings of such a guide wire roughly run along the respective path 41, 42, wherein two guide wire end pieces 45 are also indicated in FIG. 2. The precise course of the paths 41, 42 depends on the size of the windings. As is indicated in FIG. 2 by the different dimensions of the two paths 41, 42, the windings can be differently large. On account of the approach according to the invention, the windings are held in a stable manner in quite a substantial size region. The elasticity of the wound guide wire causes this to seek a circular as possible configuration, which is why from a certain winding minimal length and on account of its elasticity it is pressed outwards against the first and third guide element 11, 13 (see double arrows 50) or the third and fifth guide element 13, 15 as well as inwards against the second (double arrow 51) or fourth guide element 12, 14 and is therefore forced onto a course along the path in a stable manner.

The first path 41, which here is somewhat longer, is represented in a slightly pear-shape manner in FIG. 2, whereas the somewhat shorter second path 42 is approximately elliptical. In both cases—and this is generally the case for different embodiments of the invention—the second guide element 12 and the fourth guide element 14 can be arranged roughly in the middle-perpendicular plane between the contact points onto the first and the third contact surface 21, 23 and fourth and sixth contact surface 24, 26, respectively, and the path can therefore run roughly symmetrically to this middle-perpendicular plane. This symmetry also defines an axis 101 of the two paths 41.

Thus, with reference to FIG. 1, contact surfaces form, these being arranged such that the first path 41 is led past the first contact element at the inside (first contact surface 21), past the second guide element at the outside (second contact surface 22) and past the third guide element at the inside (third contact surface 23). Analogously, the arrangement of the contact surfaces for the second path 42 is such that the second path 42 is led past the third guide element 13 at the inside (fourth contact surface 24), past the fourth guide element 14 at the outer side (fifth contact surface 25) and past the fifth guide element 15 again at the inside (sixth contact surface 26). Considered conversely, the first, third and fifth guide element are each located at the outer side of the paths which run roughly parallel to the deposition plane (wherein the inner and outer side are defined by the curvature of the paths), so that an outer side of the respective winding abuts thereon, and the second and fourth guide element are arranged at the inside, so that an inner side of the respective windings abuts thereon.

Connecting onto the contact surfaces in the horizontal direction, the device is free of elements that project upwards from the base plate, so that the contact surfaces form a single-sided contact. The device is designed such that nothing stands in the way of a bending of the guide wire or catheter away from the contact surface in the horizontal direction.

The fact that the device is free of elements which project upwards from the base plate at each contact surface (perpendicularly away from the contact surface in the horizontal direction) is represented in FIG. 1 by way of example at the first contact surface by way of a block arrow 66. The free space (distance of possible obstacles) in the horizontal direction away from the contact surface (such as for example according to the block arrow 66) should herein be large in relation to the thickness of the winding, i.e., be a multiple of an expected winding thickness. For example, it should be at least 1 cm, at least 2 cm or at least 3 cm. In the represented embodiment example it is not limited, since no obstacle at all is arranged in the perpendicular direction away from the contact surfaces.

This configuration has the following advantages: firstly, as mentioned, differently large windings can be deposited in a stable manner, without them abutting one another or on something else. Secondly, the removal of the medical guide wire is particularly simple. One merely needs to grip a loop and press it together slightly along its smaller axis (reference numeral 47 in FIG. 2) counter to its intrinsic stiffness and then lifted. The pressing-together along the smaller axis from both sides releases the loop from all three contact surfaces, for which the fact that the contact surfaces are single-sided contact surfaces in the described sense and not, for example, a clamped holding is of significance. The removal of the guide wire also succeeds in the simplest manner by way of pressing together from only one side and subsequent lifting.

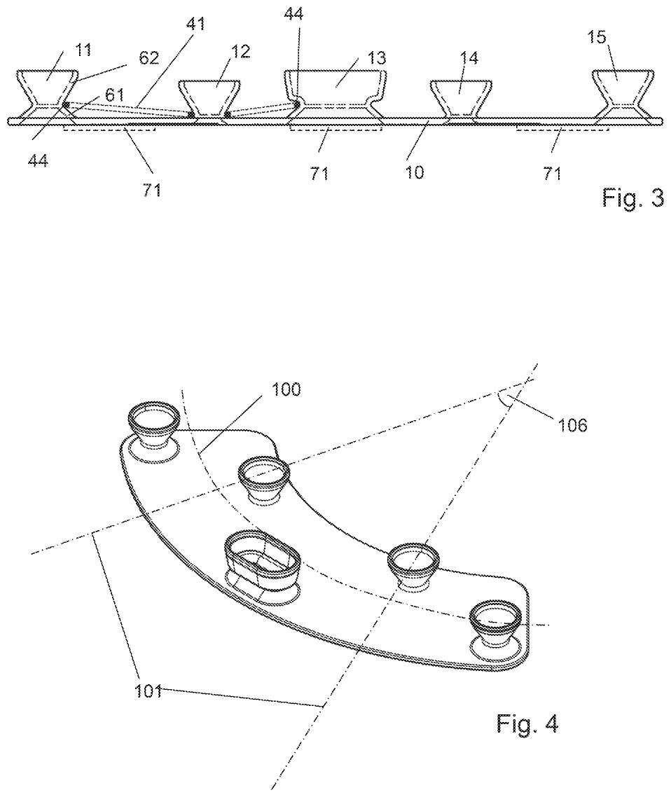

FIG. 3 shows a lateral view of the deposition device of FIGS. 1 and 2. The gripping of the guide wire can be simplified by way of the front guide elements, thus the first 11 and the third 13 as well as possibly the fifth 15 guide element including a running-out portion 61 at the lower side, i.e., towards the base plate 10, so that together with the protruding portion 62 a deepening, which is designed as a fillet, for the guide wire winding 44 is formed, the fillet being situated at a vertical distance to the base plate 10. For this reason, the guide wire at the front side can be gripped below particularly well, which greatly simplifies the operability.

Furthermore, adhesive portions 71 are schematically drawn in on the lower side of the base plate in FIG. 3. These permit the very simple fastening of the deposition device on a working table. The deposition device is advantageously positioned such that on its front side there is still sufficient depositions surface for the guide wire winding. The deposition device can also be positioned in a flat container, which includes a suitable liquid.

A further optional feature is that the base plate 10 as a whole is curved in the plane that is defined by it, such as is illustrated in FIG. 4. FIG. 4 shows the base plate with a curved axis 100. The curvature is such that the main axes 100 of the paths are at an angle 106 to one another, the angle being different than 0°, and specifically such that the axes converge towards the end-side guide element 12; 14 (corresponding to the guide element, which lies at the inside with respect to the respective path). The direction of the main axes is essentially independent of the size of the windings—which is not defined by the device—and is defined by the middle-perpendicular between the contact surfaces 21, 23; 24, 26 of the front-side guide elements 11, 13, 15, the middle-perpendicular simultaneously leading through the contact surface 22, 25 of the respective end-side guide element. The angle 106 between axes of adjacent paths is, for example, between 10° and 120° or between 20° and 120°, and it can also be up to 180° (i.e., the deposition device then defines precisely two deposition locations, lying opposite one another).

This concept of the arrangement of guide elements such that the axes of the paths form an angle to one another can also be used if more that the drawn two deposition locations (two paths) are defined, wherein a device forming a circle or an annulus includes a plurality of deposition locations that are arranged around a middle point.

The concept of the arrangements such that the axes of the paths form an angle contributes to the adjacent guide wire windings being separated from one another in a clean manner and at a sufficient distance despite a compact device and compact arrangement.

Further optional features of the deposition device that can be realised in combination with the features, which are described above or independently thereof and in combination with one another or independently of one another comprise:
The guide elements are designed in a beaker-like manner, i.e., they are hollow at the inside (cavity 31, see FIG. 1). In particular, this feature contributes to a material and weight-saving design.
The guide elements 11, 12, 14, 15, which are only assigned to a single deposition location, are roughly rotationally symmetrical about an axis perpendicular to the deposition plane, i.e., they have a round outline. This too contributes to a compact construction manner.
A middle guide element 13 forms stop surfaces 23, 24 of two adjacent deposition locations, thus serves as a common guide element for the two adjacent deposition locations. This optional feature (a guide element could also be present per deposition location) contributes to a particularly simple and compact design. As is represented in the figures, one can optionally envisage such a common guide element being wider, i.e., having a greater extension perpendicular to the direction of the paths than those guide elements that are assigned to only one deposition location.
the base plate 10 and, for example, the device as a whole can be transparent. This, for example, permits the attachment of inscriptions or the like to the lower side of the base plate, without compromising the upper side.

What is claimed is:

1. A deposition device for at least one wound medical guide wire or catheter, comprising:
a base plate with a plate upper side defining a deposition plane;
and, projecting from the upper plate side, a plurality of guide elements;
wherein the guide elements together form a first, second and third contact surface for the guide wire or catheter;
wherein the first, second and third contact surfaces are undercut, so that, in a section perpendicular to the deposition plane, they form a protrusion;
wherein the first, second and third contact surfaces are arranged and orientated such that a curved path for the wound guide wire or catheter results;
wherein the wound guide wire or catheter, when it is led along the path, abuts on the first, second and third contact surface, and
an outside of the wound guide wire or catheter lies against the first contact surface, underneath the protrusion of the first contact surface,
an outside of the wound guide wire or catheter lies against the third contact surface, underneath the protrusion of the third contact surface,
and an inside of the wound guide wire or catheter lies against the second contact surface, underneath the protrusion of the second contact surface.

2. The deposition device according to claim 1, comprising at least three guide elements projecting from the plate upper side, wherein the first contact surface is formed by a first guide element, the second contact surface is formed by a second guide element and the third contact surface is formed by a third guide element.

3. The deposition device according to claim 1, wherein the wound guide wire or catheter which is led along the path bears on the first, second and third contact surface in a point-contact-like manner.

4. The deposition device according to claim 1, wherein the contact surfaces, in a section parallel to the deposition plane, are convexly curved.

5. The deposition device according to claim 1, wherein the contact surfaces, in a section perpendicular to the deposition plane, are concavely curved.

6. The deposition device according to claim 1, wherein the guide elements forming the first and third contact surface comprise a trunk portion, a running-out portion between the trunk portion and the base plate, and an upper head portion thicker than the trunk portion on the side of the trunk portion away from the base plate, so that the guide elements forming the first and third contact surface form a recessed portion between the running-out portion and the head portion, whereby the wound guide wire or catheter is pressed, on account of its intrinsic stiffness, against the recessed portion, at a vertical distance to the plate upper side.

7. The deposition device according to claim 1, comprising a fourth, fifth and sixth contact surface for a second wound guide wire or catheter, which is led along a second curved path.

8. The deposition device according to claim 7, wherein the third and the fourth contact surface are formed by a common guide element.

9. The deposition device according to claim 8, wherein the common guide element perpendicularly to a path direction has a larger extension than guide element with only one contact surface.

10. The deposition device according to claim 7, wherein the contact surfaces are arranged in a manner that main axes of the first and second paths are
at an angle to one another, said angle being different than 0°.

11. The deposition device according to claim 1, comprising an adhesive on a plate lower side lying opposite the plate upper side, for fastening the base plate on a working surface of a working table.

12. The deposition device according to claim 1, which is free of a lateral wall that would limit a length of a winding of the medical guide wire or catheter.

13. The deposition device according to claim 1, wherein at least the base plate is transparent.

14. A use of a deposition device according to claim 1 for the deposition of a medical guide wire or catheter which is bendable counter to an elastic counter force caused by the intrinsic stiffness, wherein the guide wire or catheter is wound and is subsequently deposited such that on account of the intrinsic stiffness it abuts on the first, second and third contact surface and is pressed outwards against the first and the third contact surface and inwards against the second contact surface.
